(19) United States
(12) Patent Application Publication
(10) Pub. No.: US 2021/0065917 A1
(43) Pub. Date: Mar. 4, 2021

(54) SYSTEM AND METHOD FOR PROVIDING HIPAA COMPLIANT COMMUNICATION BETWEEN A PATIENT AND MULTIPLE HEALTHCARE ENTITIES

(71) Applicant: Muhammad Masroor, Prospect, KY (US)

(72) Inventor: Muhammad Masroor, Prospect, KY (US)

(21) Appl. No.: 16/556,512

(22) Filed: Aug. 30, 2019

(51) Int. Cl. G16H 80/00; G16H 10/60

(52) U.S. Cl. CPC G16H 80/00; G16H 10/60

(57) ABSTRACT

A system and method for providing multi-party HIPAA compliant communication includes one or more service provider interface devices, one or more medical facility interface devices, and an application for execution on a user interface device that are communicating with a platform provider system over a network. Patient information is uploaded to the platform provider system via the provider and facility interface devices. Patient family member information is uploaded by the user interface. The system verifies the uploaded patient family member is authorized to receive the patient information and provides secure textual message and document exchanges between the user interface device and each of the provider and facility interface devices performing treatment on the patient in a HIPAA compliant manner.

### TECHNICAL FIELD

The present invention relates generally to the health care industry, and more particularly to a unified communication platform providing streamlined communication between patients/family members and myriad health care facilities.

### BACKGROUND

The statements in this section merely provide background information related to the present disclosure and may not constitute prior art.

Owing to advancements in medical technologies, people are living longer than ever before and are surviving illnesses/trauma that were previously untreatable. As a result, there is an ever-increasing number of health care facilities that work together to care for the elderly and provide rehabilitative services to patients in both short term and long-term settings. Such facilities include hospitals, nursing homes, home health care providers, wound treatment centers, physical therapy centers and inpatient rehabilitation centers, for example.

Depending on the circumstances, a single patient may visit any number of these facilities during the course of treatment, or during their stay in an inpatient facility. As such, when the patient meets with a doctor or therapist, their family members and/or decision makers are not always able to attend and speak with the medical provider regarding the care for their loved one. As a result, and depending on the condition of the patient, the family must often attempt to reach the medical provider to ask questions which can be a difficult task.

Additionally, family members of patients in long term care facilities are often unaware of the activities and events happening at the facility. For example, many nursing homes have family barbecues, movie nights and other activities that are open to all residents and their families. In the past, the centers have relied upon the patients to inform family members about such events, but many such patients are unable to remember the details. As a result, family participation in these events is often low, and morale of the patients is diminished.

Although there are many known systems for allowing direct communication between a patient and a single facility, such systems are tied to the facility and do not “travel” with the patient. Stated differently, each facility/provider has a different procedure for communicating with patients and/or family members, thus requiring patients to keep track of multiple logins and communication protocols and requiring new registration of duplicate information with each new facility/provider they encounter.

Accordingly, it would be beneficial to provide a single platform that can allow a patient and/or their family to communicate with each of the patient's different health care facilities and providers that is HIPAA compliant, so as to streamline communication between the same. It would also be beneficial if the same platform contained a social media section for displaying/sharing community information about the facility in which a patient resides.

### SUMMARY OF THE INVENTION

The present invention is directed to a system and method for providing multi-party HIPAA compliant communication over a secure network. The system includes one or more service provider interface devices, one or more medical facility interface devices, and an application for execution on a user interface device that are communicating with a platform provider system over a network.

Patient information can be uploaded to the platform provider system via the provider and facility interface devices. Patient family member information can be uploaded by the user interface. The system can verify the uploaded patient family member is authorized to receive the patient information and can provide a secure mechanism for exchanging real-time two-way communications between the user interface device and each of the provider and facility interface devices.

The patient information can include any and all information pertaining to the treatment, diagnosis and/or status of a patient under the care of a participating services provider and medical facility and is HIPAA compliant.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 shows an exemplary network environment according to one embodiment of the technology.

FIG. 2 shows an exemplary flow diagram illustrating a method for allowing a healthcare entity to communicate with patient families using the system.

FIG. 3 shows an exemplary healthcare entity interface of the system of FIG. 1.

FIG. 4 shows another exemplary healthcare entity interface of the system of FIG. 1.

FIG. 5 shows another exemplary healthcare entity interface of the system of FIG. 1.

FIG. 6 shows another exemplary healthcare entity interface of the system of FIG. 1.

FIG. 7 shows another exemplary healthcare entity interface of the system of FIG. 1.

FIG. 8 shows an exemplary flow diagram illustrating a method for allowing a patient family to communicate with a plurality of healthcare entities using the system.

FIG. 9A shows an exemplary patient family interface of the system of FIG. 1.

FIG. 9B shows another exemplary patient family interface of the system of FIG. 1.

FIG. 10A shows another exemplary patient family interface of the system of FIG. 1.

FIG. 10B shows another exemplary patient family interface of the system of FIG. 1.

FIG. 11 shows another exemplary patient family interface of the system of FIG. 1.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

Identical reference numerals are used for like elements of the invention or elements of like function. For the sake of clarity, only those reference numerals are shown in the individual figures which are necessary for the description of the respective figure.

Although described throughout this document as directed to the health care industry, the inventive concepts disclosed herein are equally applicable to any number of other industries such as education, legal, manufacturing, and/or general business, for example, where secure communication between groups of individuals and their proxies is needed.

### Definitions

As described throughout this document, the term “medical providers” and “service providers” shall be used interchangeably to describe any individual, group or entity that provides services to a patient at their office or at a medical facility. Several nonlimiting examples of service providers include, but are not limited to: doctors, nurses, medical technicians, physical and speech therapists, pharmacists, dentists, optometrists, chaplains, drivers, activity organizers, and human resource individuals, for example.

The term “medical facility” and “facility” can be used interchangeably to describe a location where patients receive services from a provider. Several non-limiting examples include, but are not limited to: nursing homes, hospitals, rehabilitation centers, outpatient surgical centers, doctor offices, pharmacies, and the like.

The term “employee” can refer to any person that is employed by a participating facility or service provider office, and who communicates with patients using the system. In various embodiments, the facility employee may be a staff member or a medical provider.

The term “patient information” can include any information concerning a patient, that is to be sent to or from a facility, a medical provider or the patient themselves, using the platform. This patient information can be sent as either a voice or data communication, and can include, for example, patient reports, medical diagnosis, test results, room information, upcoming appointment scheduling, two-way chat communications, and general questions, for example.

The term “Platform provider” can include an individual or legal entity that is providing, hosting, and/or facilitating the physical platform, and/or method steps disclosed herein. In one preferred example, the Platform provider will be a duly organized company operating under the tradename JORO® and having a website that utilizes the same name. Of course, this is for illustrative purposes only, as the general platform, including any and all method steps and/or systems can be performed on any number of different websites and/or computer networks, and under any number of different names.

The below described system and method can provide a platform that functions to streamline communication between facilities, medical providers and the patients/family members under their care. As will be appreciated by one skilled in the art, aspects of the present invention may be embodied as a system, method, and/or computer program product.

Accordingly, aspects of the present invention may take the form of an entirely hardware embodiment, or an embodiment combining software and hardware aspects that may all generally be referred to herein as a “circuit,” “module,” “system”, or “feature.” Furthermore, aspects of the present invention may take the form of a computer program product embodied in one or more computer readable medium(s) having computer readable program code embodied thereon, while other aspects may utilize traditional mechanisms for conducting voice communications. In either instance, the system and other computer hardware will be necessary to facilitate the communication of patient information between care providers.

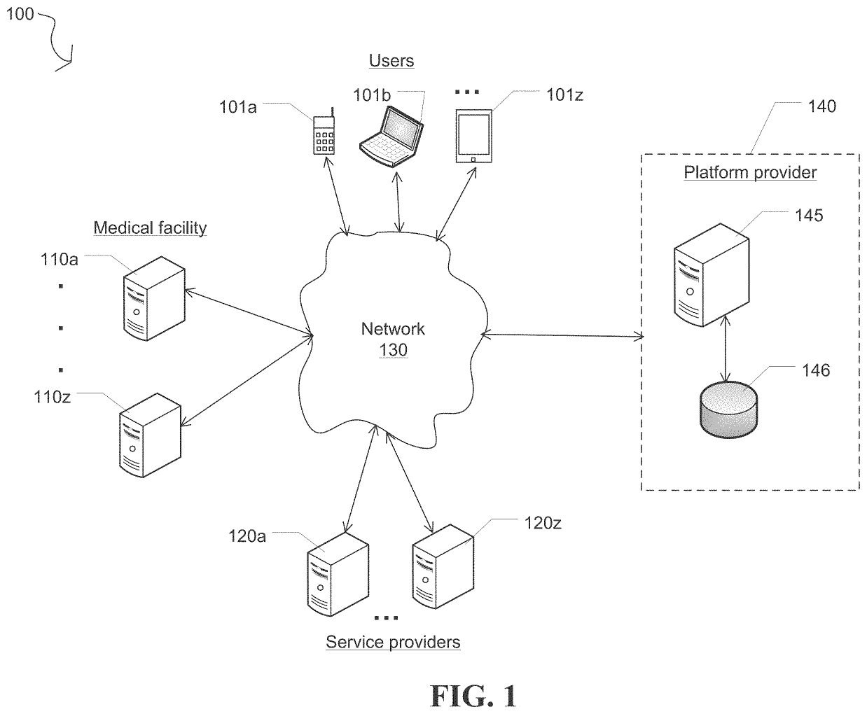

FIG. 1 is a schematic illustration of an exemplary system operating environment 100 for implementing the below described HIPAA compliant multi-party communication method. The system 100 can include, for example, a plurality of user interface devices 101a-101z, (referred to collectively at 101) for use by patients and/or designated family members, a plurality of facility interface devices 110a-110z (referred to collectively at 110); and a plurality of provider interface devices 120a-120z (referred to collectively at 120). The interface devices can be connected over a network 130 to a platform provider system 140 hosting various features of the below described methodology.

The platform provider system 140, according to one embodiment, can include one or more individual computing devices 145 that can be connected to one or more databases 146 on which various portions of the method can be performed. The system 140 can function to provide a central hub for controlling the communication between the various user interfaces 101, 110 and 120, through any number of different mediums such as the above noted website, for example. In this regard, one or more of the individual computing devices described herein as the platform provider system can comprise a web server, an email server, a communication server, and so forth, or the system can employ a single server-type device which functions to handle each of these processes.

Portions of the below described method can be implemented as a computer program product, i.e., a computer program tangibly embodied in a non-transient machine-readable storage device, for execution by, or to control the operation of, a data processing apparatus. The computer program can be written in any form of computer or programming language, including source code, compiled code, interpreted code, scripting code (e.g., JavaScript) and/or machine code, and the computer program can be deployed in any form, including as a stand-alone program or as a subroutine, element, or other unit suitable for use in a computing environment.

The database 146 can include one or more independent storage devices that can function to receive and store the patient information along with any other form of information. In one embodiment, the database can function to receive and store facility information, provider information and/or patient information. As described herein, the database 146 can include various types of computer-readable storage mediums, such as, for example, semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. In addition, the devices can be operatively coupled to a communications network, such as network 130, to receive instructions and/or data from the network and/or to transfer instructions and/or data to the network.

As described herein, each of the interface devices 101, 110 and 120 can include any type of processor-enabled device such as a computer, tablet, or smart phone, for example, that can be operated by a human user. Moreover, each of the interface devices can also include one or more client applications, such as a web browser, or mobile application (i.e., App), for example that can be installed on the device in order to allow a device user to communicate with and view content from other devices over the network 130.

Owing to the sensitive nature of the information to be shared across the system, and in order to be HIPAA compliant, each of the facility devices 110, provider devices 120, and one or more of the platform devices 140 can preferably be constructed as a purpose-built device having dedicated and password protected internal or external storage mediums. These purpose-built devices can further include secure network interfaces having an embedded random number generator which can be synced across each system device. Such a feature can ensure that the purpose-built non-generic processor enabled devices perform the below described methodology in a completely secure manner that cannot be achieved through the use of off-the-shelf hardware. Such a feature being necessary to satisfy the rigid HIPAA guidelines for confirming the identity of individuals who receive sensitive patient information.

In various embodiments, the network 130 is a transmission medium that facilitates any form or medium or digital or analog communication (e.g., a communication network). Transmission mediums can include one or more packet-based networks and/or one or more circuit-based networks in any configuration. Packet-based networks can include, for example, the Internet, a carrier internet protocol (IP) network (e.g., local area network (LAN), and/or a wide area network (WAN). Circuit-based networks can include, for example, the public switched telephone network (PSTN), a wireless network (e.g., RAN, code-division multiple access (CDMA) network, time division multiple access (TDMA) network, global system for mobile communications (GSM) network), Bluetooth, WiFi, or Personal Area Networks (PANs), Near Frequency Communication (NFC) network, and/or other circuit-based networks.

Information transfer over the network 130 can be performed by a communication module based on one or more communication protocols. Communication protocols can include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), a Global System for Mobile Communications (GSM) protocol, a Push-to-Talk (PTT) protocol, a PTT over Cellular (POC) protocol, a Real-time Messaging protocol (RTMP), a Real-time Media Flow Protocol (RTMFP) and/or other communication protocols.

The following method for providing HIPAA compliant communication between a patient and multiple healthcare entities (e.g., facilities and providers) is designed to eliminate the need for patients to have to access a different system for each entity by providing a single integrated platform that is secure, identity verified and maintains a complete log of all communications as is required by HIPAA. The method is also designed to allow a designated contact person such as a relative or those with power of attorney over the patient (e.g., patients family) to communicate with the healthcare entities to oversee the care their loved ones are receiving.

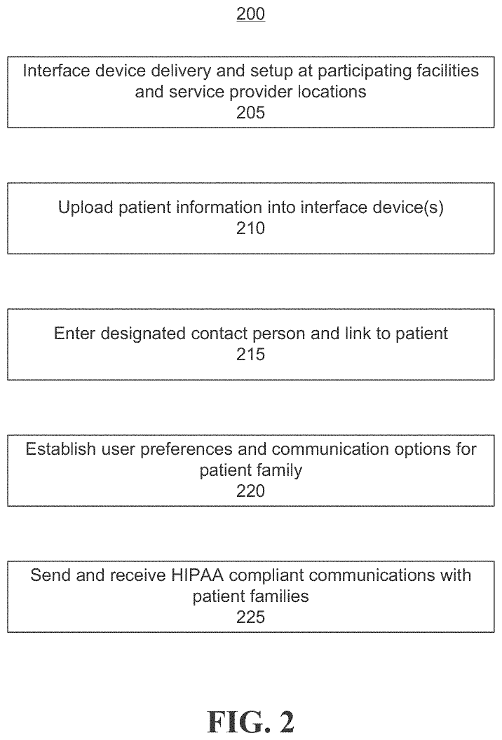

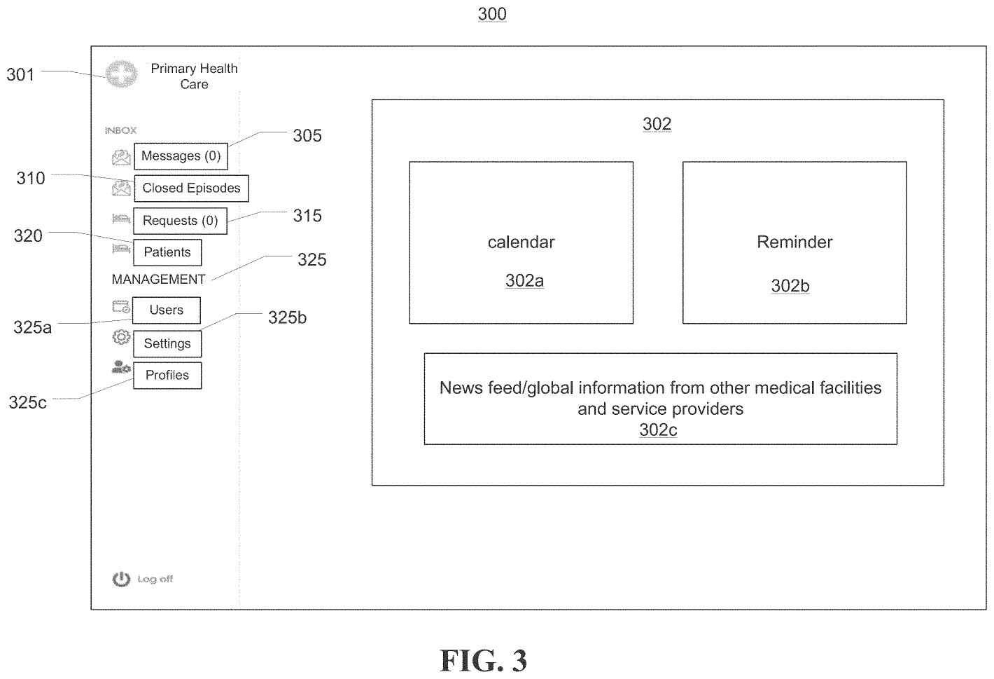

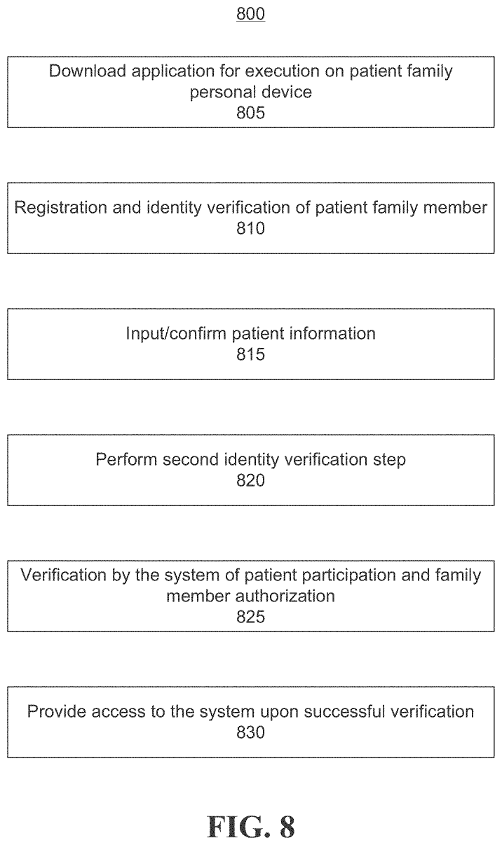

A method for providing multi-party HIPAA compliant communication utilizing the network system 100 will now be described with respect to FIGS. 2 and 8. Moreover, several exemplary presentation screens which can be generated by the system are presented with respect to FIGS. 3-7 and FIGS. 9-11. Although described below with respect to particular steps and screens, this is for illustrative purposes only, as the methodology described herein can be performed in a different order than shown, and the presentation screens can include any number of additional information and features.

FIG. 2 illustrates an exemplary flow chart method 200 for allowing a healthcare entity to communicate with patient families using the system 100. The method begins at step 205 with the installation of one or more interface devices 110 and 120 at each participating medical facility and service provider location, respectively. The installation procedure includes connecting each of the user interface devices to the platform provider 140 over the network 130, and ensuring each device can communicate in a secure manner according to HIPAA standards.

Once the installation procedure has completed, the method can proceed to step 210, wherein patient information on file at the particular facility or provider can be uploaded into the system and stored in the secure database 146. The amount and type of patient information may vary depending on a number of factors, but at a minimum the patients' name, birthdate and contact information (e.g., telephone, fax, email, physical address) should be provided. In some instances, the facility may upload the entire patients file for secure storage and retrieval using the system platform.

Next, the method can proceed to step 215, wherein each patient's designated contact person such as a relative or individual with power of attorney that has been authorized by the patient to send and receive patent information can be entered into the system and linked to the patient. By pre-establishing a designated contact person who will have a discrete and secure login access to the platform, the system advantageously performs the required identity verification thus eliminating the need for facilities and providers to individually verify the identity of a person requesting patient information each time. Such a feature provides a streamlined communication pathway that remains fully HIPAA compliant.

Next, the method can proceed to step 220 wherein employees from the particular facility or provider can be given options for establishing or changing user preferences and updating system settings. Finally, the method can proceed to step 225 wherein the employee can begin exchanging secure messages containing patient information with a patient or their designated contact (referred to collectively as a “patient family”) in a HIPAA compliant manner using the secure system 100.

FIGS. 3-7 present exemplary user interfaces of the system 100 of FIG. 1, for display to participating medical facilities and service providers demonstrating exemplary HIPAA compliant communications with a patient family. In one embodiment, the below illustrated presentation screens can be generated by the platform provider 140 and displayed on one or more of the interface devices 110 and 120. Although illustrated in the form of a website and website pages, this is for illustrative purposes only, as the inventive concepts disclosed herein can be implemented across a wide variety of different platforms without deviating from the scope and spirit of the inventive concepts disclosed herein.

FIG. 3 illustrates an exemplary home presentation screen 300 that can be generated by the platform provider 140 to be displayed on a particular facility or provider interface device 110 and 120, respectively. In this example, the home presentation screen 300 for the particular facility or provider 301 can be generated in response to an employee logging onto the JORO system via a secure website URL, for example.

As shown, the screen 300 can include, a user-customizable section 302 which can display, for example, various calendars 302a, reminders 302b, and/or messages 302c from other medical facilities or service providers, along with options for sending and receiving new messages with established patient families 305, a listing of previously sent messages 310, a listing of new patient family requests 315, a listing of all client patients 320 and a plurality of system management options 325 such as establishing and/or updating employee users 325a, adjusting internal presentation screen settings 325b and/or adjusting the facility or provider profile 325c that is visible to all patient families, medical facilities and service providers on the entire platform.

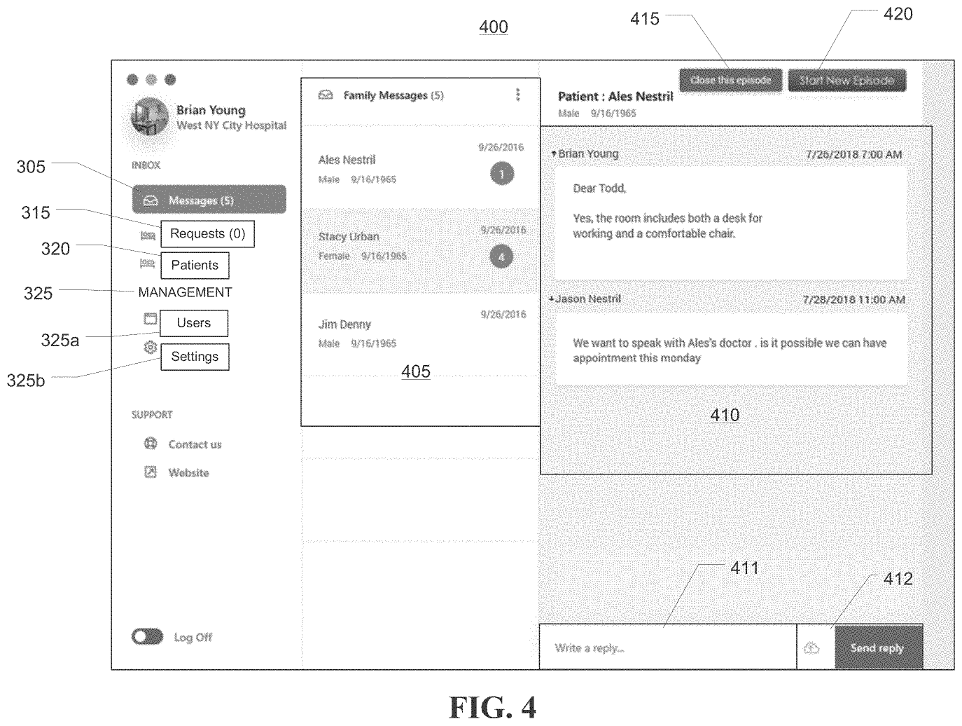

FIG. 4 illustrates one embodiment of an exemplary Messages presentation screen 400 which can be generated by the platform 140 in response to an employee selecting the Messages tab 305 from the home presentation screen 300. As shown, the Messages screen 400 provides a mechanism for the employee to send and receive textual messages with patient families. As will be explained below, because the patient family must be pre authorized by the patient, and verified by the healthcare entity before messages can be sent or received, the employee does not need to individually verify the identity of the sender of each message they receive, as any messages contained in this section have been fully vetted.

In one embodiment, the messages screen can include a listing of open conversations 405, and upon selecting a particular patient from the list, a new window 410 can be generated. The window 410 can include all communications to and from the patient family for a particular episode. A message box 411 can be provided to allow the employee to respond to the patient family questions and to upload 412 files such as medical documents, images and the like.

As described herein, an “episode” shall refer to a series of messages that are sent and received concerning a particular issue regarding a patient. Once the particular issue has been resolved, the episode will be closed 415 by the employee and the communications will be stored by the platform database in accordance with HIPAA standards. Moreover, should the employee wish to initiate contact with the patient family, the employee can start a new episode by clicking box 420.

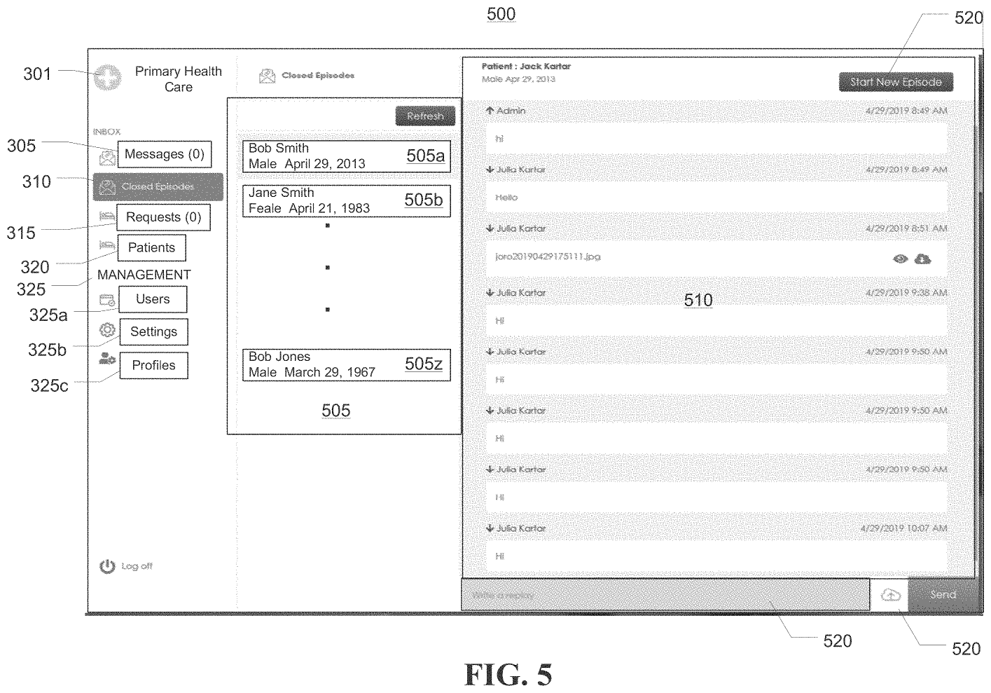

FIG. 5 illustrates one embodiment of an exemplary Closed Episodes presentation screen 500 which can be generated by the platform 140 in response to an employee selecting the Closed Episodes tab 310 from the home presentation screen 300. As shown, the screen 500 provides a mechanism for the employee to view all previous communications with patient families that have been closed as described above.

In one embodiment, the screen 500 can include a list of patients 505 for which previous communications (i.e., closed episodes) using the system have been made. Upon selecting a particular patient 505a-505z from the list, a new window 510 can be generated. The window 510 can include each of the actual communications to and from the patient family, by episodes. As such, if a patient has a heart attack, for example, the facility employee can search the records for any communications that particularly related to cardiac issues. Such a feature is incredibly useful, as it eliminates the need for the employee to manually scroll through an entire patient file, and instead organizes everything pertaining to such an episode.

If the employee needs to communicate with a particular patient family about this or any other episode, they can select the Start new Episode tab 520, type a message in the message box 511 and upon clicking send 512, the new message will be sent to the patient family and listed in the active Messages presentation screen 400 as an existing or new episode, respectively.

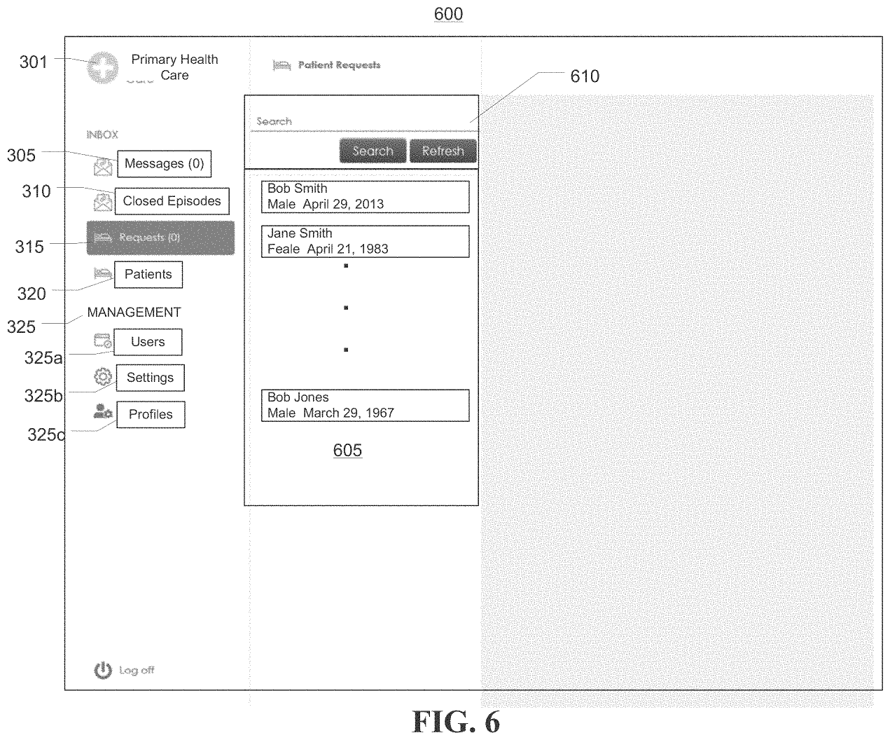

FIG. 6 illustrates one embodiment of an exemplary new patient family Request presentation screen 600 which can be generated by the platform 140 in response to an employee selecting the Requests tab 315 from the home presentation screen 300. As shown, the screen 600 provides a mechanism for a facility or provider employee to view, approve and/or deny requests from patient families to establish communication with the particular facility or provider 330.

In one embodiment, the screen 600 can include a listing of all active requests 605 that have been sent by patient families for a particular patient to the facility 301. The employee can also search 610 through the requests 605 using information such as the patients name, birthdate social security number, or other information. In either instance, upon selecting a particular name 605a-605z, the screen will provide the employee with the option to approve or deny the request.

Although the specific protocols for approving or denying patient requests can vary based on the individual policies of the facility or provider, the system will provide the employee with options for cross referencing patient requests with established patients and/or to view a list of patient family members that have been pre-authorized to send and receive patient information.

In either instance, if the employee approves the request, the selected patient family will receive a notification, and the patient family will appear in the listing of patients, whereby two way communication using the system will be permitted. If the employee denies the request for any reason, the patient family will receive a notification and the system will not allow messages to be sent.

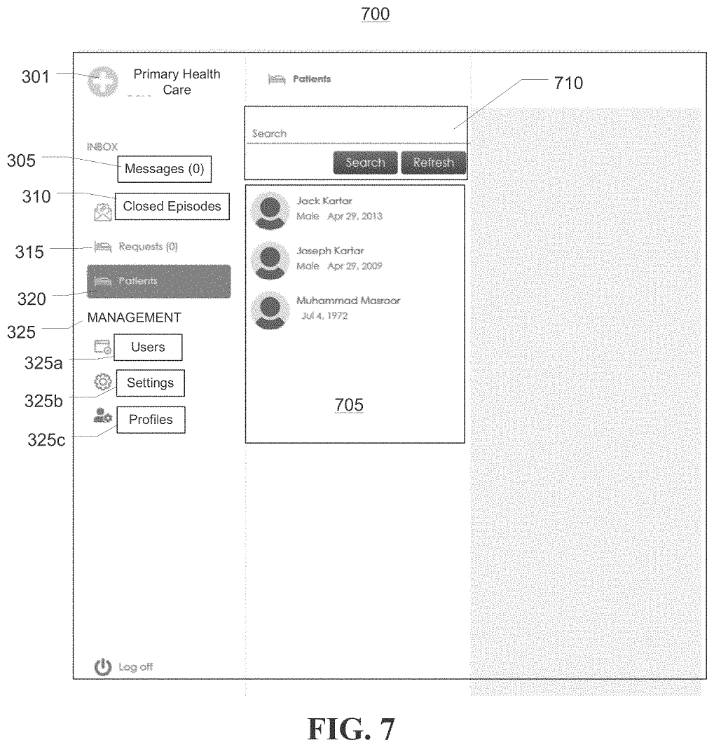

FIG. 7 illustrates one embodiment of an exemplary List of Patients presentation screen 700 which can be generated by the platform 140 in response to an employee selecting the Patients tab 320 from the home presentation screen 300. As shown, the screen 700 provides a mechanism for a facility or provider employee to retrieve and view all patients 705 who are participating in the communication platform and system 100 and that are under the care of the particular facility or provider 330.

The employee can also search 710 through the listing 705 using information such as a particular patients name, birthdate, social security number, patient family name (if a communication request has been received) and/or other information. In either instance, upon selecting a particular name, the screen 700 will provide the employee with the option to view contact details for the patient, patient files, records, images, and/or to initiate a new episode and conduct HIPAA compliant written communications as described above.

Although not specifically illustrated, the system 100 can include functionality for allowing healthcare entities to profit by charging a subscription to patient families. The subscription charges can function to offset the cost of user identity verification that will be described below, for example. Of course, the system can also provide options for allowing healthcare entities to provide advertisements to all system users which may reduce or eliminate any subscription charges.

Now that the system has been described from the medical facility and service provider standpoint, a description of the use and operation of the system by a patient family will be provided.

FIG. 8 illustrates one exemplary flowchart method 800 for allowing a patient family to communicate patient information with a plurality of medical facilities and service providers. The method can begin at step 805 wherein the patient family can download a mobile application (e.g., the JORO mobile application) for installation on their user interface device 101 such as a smartphone or other processor enabled device. In the preferred embodiment, the App will communicate over the network using 130 using secure and encrypted protocols. Of course, the method and system is not limited to the use of a mobile application and smartphone, as any number of other devices and communication protocols are contemplated.

Next, the method can proceed to step 810 where the patient family member can perform an initial registration and identity verification process by providing information such as their name, birthdate, driver license, credit card and the like. Next, the method can proceed to step 815 wherein the family member can input information about the patient for whose information they are seeking. This may include providing the patient's name, birthdate, and/or social security number, for example.

Next, the method can proceed to step 820 where the system can perform a second identity verification step by requiring the patient family member to enter a patient-specific code or other token that has been provided to the patient family member by the patient themselves and/or a healthcare entity who has physically verified the person's identity.

Next, the method can proceed to step 825, where the system can verify that the requested patient is under the care of a medical facility or service provider participating in the platform and system 100, and that the code provided at step 820 is valid for the listed patient. If the verification is not successful, the method can terminate, and the family member will not be granted access to the system.

Upon successful verification, the method can proceed to step 830 where the patient family member will be granted access to the system 100 via the App on their user interface device 101. Upon being granted access and undergoing a third verification step (described below) the patient family will be able to send and receive patient information in accordance with HIPAA standards with all participating medical facilities and service providers attending to the care of the selected patient.

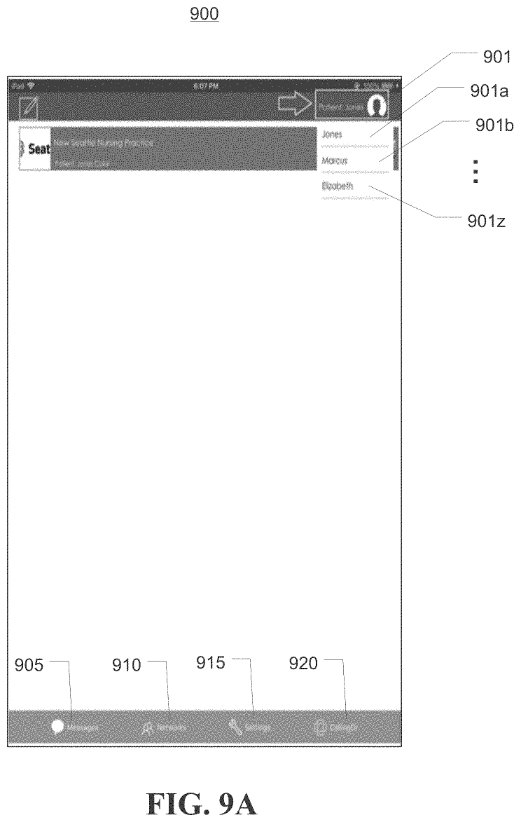

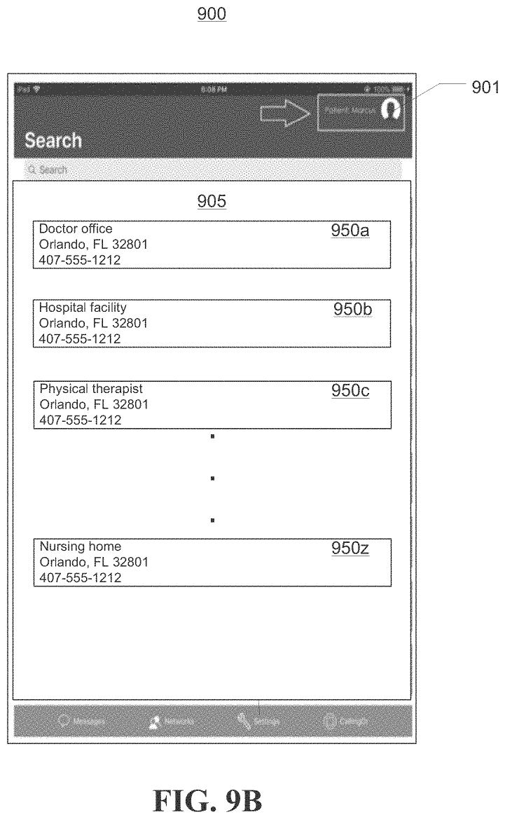

FIGS. 9-11 present exemplary user interfaces of the system 100 of FIG. 1, for display to patient family when conducting HIPAA compliant communications with a medical facility or service provider. In one embodiment, the below illustrated presentation screens can be generated by the platform provider 140 and displayed on the user interface device 101. Although illustrated in the form of a mobile application and smartphone, this is for illustrative purposes only, as the inventive concepts disclosed herein can be implemented across a wide variety of different platforms without deviating from the scope and spirit of the inventive concepts disclosed herein.

FIG. 9A illustrates an exemplary home presentation screen 900 that can be generated by the platform provider 140 to be displayed on a user interface device 101. In this example, the home presentation screen 900 can include a patient tab 901 listing each patient 901a-901z for whom the patient family is authorized to access. A main body section 905, a Messages tab 910, a Networks tab 915, a settings tab 920 and a Calling Dr. Tab 925 are also provided.

As shown at FIG. 9B, upon selecting a patient name from tab 901, the main body section of the presentation screen 900 can populate with a list 950a-950z of each participating medical facility and service provider that is attending to the selected patient. Each of the listed entities may be selected by the user to initiate contact.

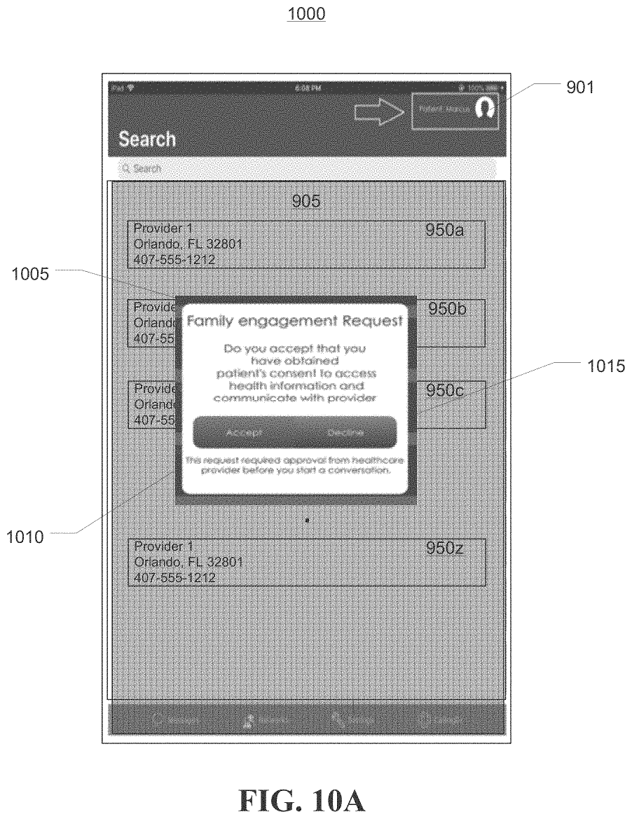

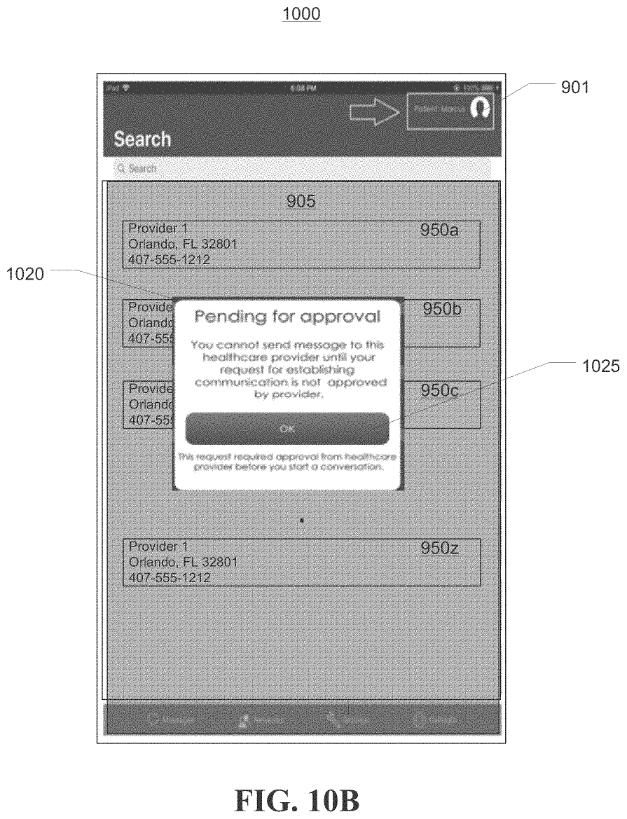

FIG. 10A illustrates one embodiment of an exemplary confirmation screen 1000 which can be generated by the platform 140 in response to a user initiating first contact with an entity from the list. As shown, the screen 1000 can require the patient family member to explicitly confirm 1010 they have authorization to initiate the request and receive patient information for the selected patient with the selected healthcare entity. If they do not have such permission, the user will select Decline 1015.

As shown at FIG. 10B, upon selecting Accept 1010, the platform 140 can send the request to the selected entity and the screen 1000 can display a second notification 1020 that the request has been sent and is awaiting approval. Upon acknowledging receipt of the message 1025, the user will be returned to the home screen pending the outcome of the facility verification. As noted above at FIG. 6, such requests will be displayed to the selected entity in the listing of patient requests 605 whereby the healthcare entity will confirm the patient family member has been authorized by the patient (e.g., a third verification step) and/or meets whatever facility specific protocols are established.

Upon approval by the selected entity and/or upon previously being approved by the selected entity, the user will be able to send and receive patient information with the entity about the designated patient using the system 100.

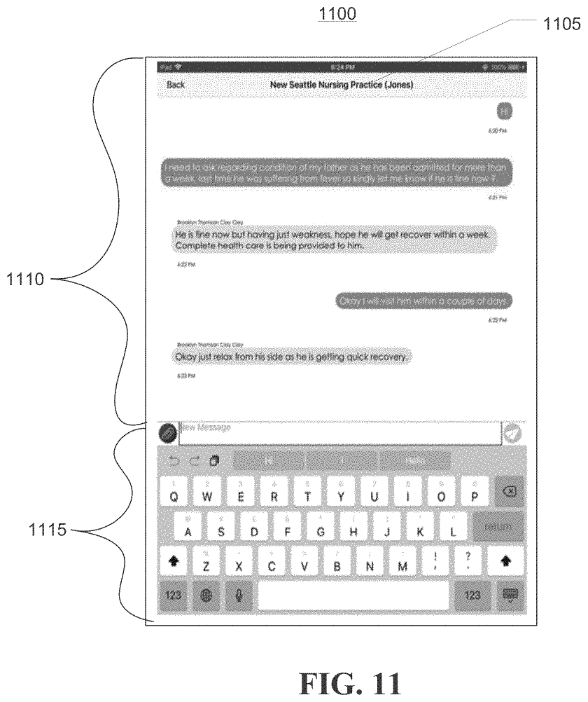

In this regard, FIG. 11 illustrates one embodiment of an exemplary user Messages presentation screen 1100 which can be generated by the platform 140 in response to an approved patient family selecting the Messages tab 905 or directly selecting an entity. The Messages screen 1100 provides a mechanism for the patient family to send and receive textual messages with medical facilities and services providers.

As shown, the messages screen 1100 can include an indication of the entity 1105 with whom the patient family is speaking, along with a chat window 1110 showing all communications to and from the entity for a particular episode. A message box and keyboard 1115 can be provided to allow the user to ask questions, upload documents and respond to questions from the entity employee. The messages will be displayed at the entity on the open conversations window 410 as described above at FIG. 4.

Because the identity of the patient family has been first verified by the system (e.g., step 810), and subsequently verified by the patient (e.g., steps 820-825) and the healthcare entity e.g., (FIG. 6), the system advantageously provides triple redundancy verification to ensure the patient information is being sent only to an authorized patient family member. Such features exceed the stringent HIPAA requirements while simultaneously reducing the workload of facility employees by eliminating the need to independently verify each request for patient information by a family member.

Accordingly, the above described system and method provides a secure and HIPAA compliant communication and document storage facility that allows communications between a patient family and each healthcare facility attending to a particular patient, without requiring independent verification by each entity with every request.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As will be appreciated by one skilled in the art, aspects of the present invention may be embodied as a system, method or computer program product. Accordingly, aspects of the present invention may take the form of an entirely hardware embodiment, or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Although described above as utilizing specific components, features and/or method steps, those of skill in the art will recognize that many modifications can be performed without deviating from the scope and spirit of the inventive concepts disclosed herein. Accordingly, the above description is not intended to be limiting upon the invention in any way.

What is claimed is:

1. A method for providing multi-party HIPAA compliant communication over a network, said method comprising:
installing a medical facility interface device at a medical facility;
installing a provider interface device at a service provider;
providing a mobile application for installation on a user interface device;
establishing communication between each of the provider interface device, the medical facility interface device, and a platform provider system;
receiving, via the user interface device, identification information about each of a patient and a patient family member; and
sending and receiving secure communications containing patient information via the user interface device and each of the medical facility interface device, and the provider interface device.

2. The method of claim 1, wherein the identification information includes uploading identification documents of the patient family member via the user interface device.

3. The method of claim 2, wherein the identification documents include, at least one of a driver license photograph or a credit card information.

4. The method of claim 2, wherein the identification documents include each of a driver license photograph and a credit card information.

5. The method of claim 1, further comprising:
verification, via the platform provider system that the patient family member is authorized by the patient to receive the patient information.

6. The method of claim 1, further comprising:
displaying, via the user interface device, a list of each medical facility and service provider that is treating the patient.

7. The method of claim 6, further comprising:
receiving, via the user interface device, a request to communicate patient information with one of the listed medical facility and service provider.

8. The method of claim 7, further comprising:
accepting, via one of the medical facility interface device and the service provider interface device the request to communicate patient information.

9. The method of claim 1, further comprising:
storing, via the platform provider system, each of the secure communications containing patient information.

10. The method of claim 9, further comprising:
providing, via each particular facility interface device access to each of the stored secure communications sent or received from the particular facility interface device.

11. The method of claim 9, further comprising:
providing, via each service particular provider interface device access to each of the stored secure communications sent or received from the particular service provider interface device.

12. The method of claim 1, wherein one or more of the provider interface device, the medical facility interface device and the platform provider system are purpose-built machines designed and configured solely to execute method steps for providing multi-party HIPAA compliant communication.
